(19) United States
(12) Patent Application Publication
(10) Pub. No.: US 2021/0315545 A1
(43) Pub. Date: Oct. 14, 2021

(54) ULTRASONIC DIAGNOSTIC APPARATUS AND ULTRASONIC DIAGNOSTIC SYSTEM

(71) Applicant: CANON MEDICAL SYSTEMS CORPORATION, Otawara-shi (JP)

(72) Inventors: Atsuko SUGIYAMA, Nasushiobara (JP); Guang Yi ONG, Nasushiobara (JP)

(73) Assignee: CANON MEDICAL SYSTEMS CORPORATION, Otawara-shi (JP)

(21) Appl. No.: 17/224,227

(22) Filed: Apr. 7, 2021

(30) Foreign Application Priority Data
Apr. 9, 2020 (JP) 2020-070324

(51) Int. Cl. A61B 8/08; A61B 8/00; G06N 20/00

(52) U.S. Cl. CPC A61B 8/5207; A61B 8/54; G06N 20/00; A61B 8/46; A61B 8/4444

(57) ABSTRACT

An ultrasonic diagnostic apparatus of embodiments includes processing circuitry. The processing circuitry is configured to convert a signal into image information, the signal being generated by an ultrasonic probe receiving reflected waves of ultrasonic waves which have been transmitted from the ultrasonic probe and reflected from a subject, acquire information representing a relative relationship of the ultrasonic probe with respect to the subject, acquire at least one of information representing subject characteristics of the subject and information representing apparatus characteristics of the ultrasonic diagnostic apparatus, and generate an operation candidate for the ultrasonic probe on the basis of the acquired information representing the relative relationship of the ultrasonic probe with respect to the subject and the acquired at least one of the information representing the subject characteristics and the information representing the apparatus characteristics.

### CROSS-REFERENCE TO RELATED APPLICATION

The present application claims priority based on Japanese Patent Application No. 2020-070324, filed Apr. 9, 2020, the content of which is incorporated herein by reference.

### FIELD

Embodiments disclosed in the present specification and drawings relate to an ultrasonic diagnostic apparatus and an ultrasonic diagnostic system.

### BACKGROUND

There is an ultrasonic diagnostic system including an ultrasonic probe and an ultrasonic diagnostic apparatus. The ultrasonic probe transmits ultrasonic waves to a subject and outputs information based on reflected waves reflected by the subject to the ultrasonic diagnostic apparatus as reflected wave information. The ultrasonic diagnostic apparatus converts the reflected wave information output from the ultrasonic probe into image information and displays the image information. An operator who performs diagnosis on the subject by operating the ultrasonic diagnostic apparatus diagnoses the presence or absence of a lesion in the subject, and the like while viewing an image displayed by the ultrasonic diagnostic apparatus, for example.

There are cases where the shape and the size of a lesion included in image information change, for example, according to a positional relationship between a subject and the ultrasonic probe. In this case, there are cases where the shape and the size of the lesion is not able to be correctly recognized and thus a diagnosis becomes difficult. With respect to this problem, there is an ultrasonic diagnostic apparatus including a sensor that detects a relative position between the subject and the ultrasonic probe, and the like in real time.

However, even if a relative position between the subject and the ultrasonic probe, and the like are detected in real time, there are cases where the shape of a lesion changes due to pressure when the ultrasonic probe is pressed against the subject and the direction thereof.

### BRIEF DESCRIPTION OF THE DRAWINGS

FIG. 1 is a block diagram of an ultrasonic diagnostic system 1 of a first embodiment.

FIG. 2 is a diagram showing a state in which diagnosis is being performed on a subject H using the ultrasonic diagnostic system 1.

FIG. 3 is a flowchart showing an example of processing of an ultrasonic diagnostic apparatus 100 of the first embodiment.

FIG. 4 is a diagram showing an example of a page displayed on a display device 42.

FIG. 5 is a block diagram of an ultrasonic diagnostic system 2 of a second embodiment.

FIG. 6 is a flowchart showing an example of processing of an ultrasonic diagnostic apparatus 200 of the second embodiment.

FIG. 7 is a conceptual diagram showing a data flow through which the ultrasonic diagnostic apparatus 200 performs machine learning and executes a normal diagnosis.

FIG. 8 is a flowchart showing an example of processing of an external apparatus.

FIG. 9 is a conceptual diagram showing a data flow through which the external apparatus 220 performs machine learning and the ultrasonic diagnostic apparatus 200 executes a normal diagnosis.

FIG. 10 is a block diagram of an ultrasonic diagnostic system 3 of a third embodiment.

FIG. 11 is a diagram showing the appearance of the ultrasonic diagnostic system 3.

FIG. 12 is a diagram showing an example of a page displayed on the display device 42.

### DETAILED DESCRIPTION

An ultrasonic diagnostic apparatus of embodiments includes processing circuitry. The processing circuitry is configured to convert a signal into image information, the signal being generated by an ultrasonic probe receiving reflected waves of ultrasonic waves which have been transmitted from the ultrasonic probe and reflected from a subject, acquire information representing a relative relationship of the ultrasonic probe with respect to the subject, acquire at least one of information representing subject characteristics of the subject and information representing apparatus characteristics of the ultrasonic diagnostic apparatus, and generate an operation candidate for the ultrasonic probe on the basis of the acquired information representing the relative relationship of the ultrasonic probe with respect to the subject and the acquired at least one of the information representing the subject characteristics and the information representing the apparatus characteristics.

Hereinafter, ultrasonic diagnostic apparatuses and ultrasonic diagnostic systems of embodiments will be described with reference to the drawings.

### First Embodiment

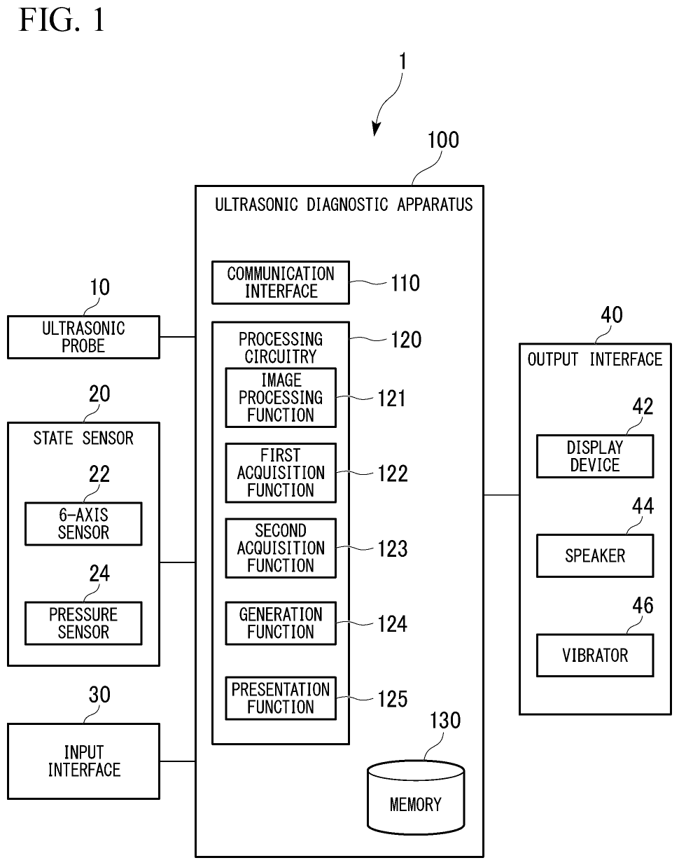

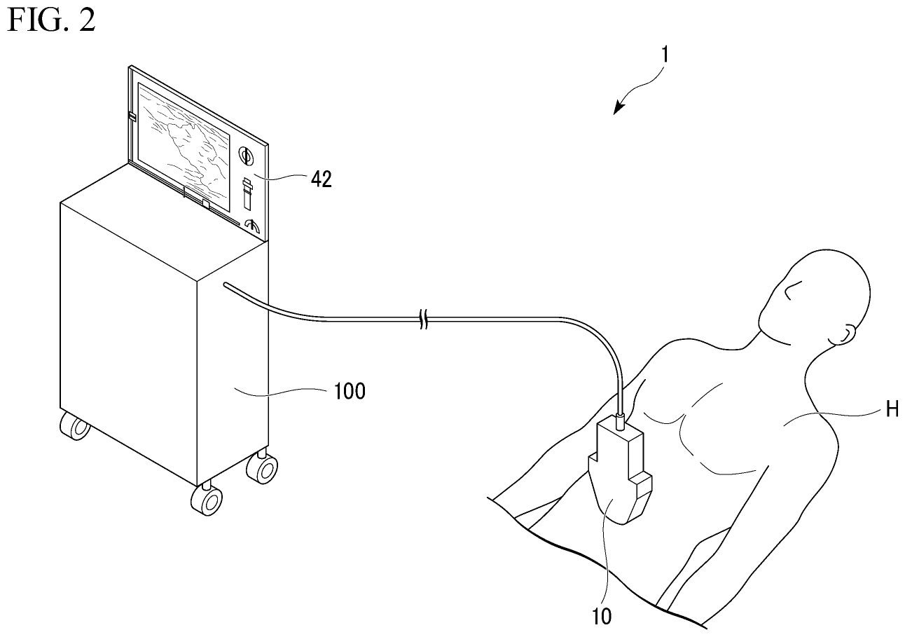

FIG. 1 is a block diagram of an ultrasonic diagnostic system 1 of the first embodiment and FIG. 2 is a diagram showing a state in which a diagnosis is being performed on a subject H using the ultrasonic diagnostic system 1. As shown in FIG. 1, the ultrasonic diagnostic system 1 includes, for example, an ultrasonic probe 10, a state sensor 20, an input interface 30, an output interface 40, and an ultrasonic diagnostic apparatus 100. As shown in FIG. 2, in the ultrasonic diagnostic apparatus 100, a display device 42 is provided in the output interface 40.

The ultrasonic probe 10 is pressed to an examination target region of a subject H, for example, on the basis of a manual operation of an operator that is not shown. For example, the ultrasonic probe 10 transmits ultrasonic waves to the subject in order to acquire an image of the inside of the body of the subject H. The ultrasonic probe 10 receives reflected waves of the transmitted ultrasonic waves. The ultrasonic probe 10 generates reflected wave information that is a signal (echo signal) of reflected waves of the ultrasonic waves generated according to reception through a transmission/reception surface and outputs the reflected wave information to the ultrasonic diagnostic apparatus 100.

As shown in FIG. 1, the state sensor 20 includes, for example, a 6-axis sensor 22 and a pressure sensor 24. The 6-axis sensor 22 and the pressure sensor 24 are provided in the ultrasonic probe 10, for example. The state sensor 20 detects a relative position, a scanning direction, a rotational direction, an inclination, and a pressure when the ultrasonic probe 10 is pressed to a subject (hereinafter referred to as a “pressing pressure”) as a state that is a relative relationship between the ultrasonic probe 10 and the subject. The state of the ultrasonic probe 10 with respect to the subject may be detected by a sensor other than the state sensor 20.

The 6-axis sensor 22 is, for example, a sensor that detects a 3-axis acceleration and a 3-axis angular speed. The 6-axis sensor 22 detects a relative position, a scanning direction, a scanning speed, a rotational direction (rotating speed), and an inclination (direction) of the ultrasonic probe 10 with respect to the subject on the basis of the detected 3-axis acceleration and 3-axis angular speed. For example, the 6-axis sensor 22 detects an acceleration in each direction in three dimensions and calculates a difference between a known position, for example, a default position, and a current position. The 6-axis sensor 22 detects a relative position and a scanning direction of the ultrasonic probe 10 with respect to the subject on the basis of the calculated position difference. To detect the relative position and the scanning direction, a 3-axis sensor instead of the 6-axis sensor 22 may be used.

The relative position of the ultrasonic probe 10 with respect to the subject may be detected through other methods. For example, a relative position sensor may include a camera that images the subject. In this case, the relative position sensor detects the relative position of the ultrasonic probe 10 with respect to the subject through optical difference identification using an image captured by the camera, for example. The relative position sensor may be a sensor using an electromagnetic method.

The 6-axis sensor 22 detects a current position of the ultrasonic probe 10, for example, on the basis of the 3-axis acceleration. The 6-axis sensor 22 calculates a scanning direction of the ultrasonic probe 10, for example, by calculating a difference between the current position of the ultrasonic probe 10 and a known position (default position). The 6-axis sensor 22 calculates the scanning speed of the ultrasonic probe 10, for example, on the basis of a rate of change in the scanning direction of the ultrasonic probe 10. A scanning direction and an operating speed of the ultrasonic probe 10 may be obtained using a 3-axis sensor that detects a 3-axis acceleration.

The 6-axis sensor 22 detects a rotational direction of the ultrasonic probe 10, for example, on the basis of the 3-axis angular speed. The 6-axis sensor 22 calculates the rotational direction of the ultrasonic probe 10, for example, by calculating a difference between a current angle of the ultrasonic probe 10 and a known angle (default angle). The 6-axis sensor 22 calculates a rotating speed of the ultrasonic probe 10, for example, on the basis of a rate of change in the rotational direction of the ultrasonic probe 10. The 6-axis sensor 22 outputs information on the detected relative position, scanning direction, scanning speed, rotational direction (rotating speed), and an inclination (orientation) corresponding to a state of the ultrasonic probe 10 with respect to the subject to the ultrasonic diagnostic apparatus 100.

The pressure sensor 24 is formed of a conductive film having a piezoelectric layer formed on the inside thereof, for example. For example, the pressure sensor 24 includes two outer electrodes on the outside and an inner electrode sandwiched between the two outer electrodes. The pressure sensor 24 measures a current value of current flowing between the two outside electrodes when a pressure is applied between the two outside electrodes. The pressure sensor 24 detects a pressure applied to the pressure sensor 24, in other words, a pressure applied between a subject and the ultrasonic probe 10 on the basis of the measured current value. The pressure sensor 24 outputs information on the detected pressure to the ultrasonic diagnostic apparatus 100. In the following description, information on a state of the ultrasonic probe 10 with respect to a subject will be referred to as “probe state information.”

The 6-axis sensor 22 may detect a 3-axis acceleration and a 3-axis angular speed of the ultrasonic probe 10. The pressure sensor 24 may detect a measured current value. In this case, the state sensor 20 outputs the 3-axis acceleration and the 3-axis angular speed of the ultrasonic probe 10 detected by the 6-axis sensor 22 and detection information on the current value measured by the pressure sensor 24 to the ultrasonic diagnostic apparatus 100. The ultrasonic diagnostic apparatus 100 calculates probe state information on the basis of the output detection information.

The input interface 30 includes, for example, physical operating parts such as a mouse, a keyboard, and a touch panel. The input interface 30 outputs subject information such as details stored in a hospital information system (HIS) and details written on medical questionnaires to the ultrasonic diagnostic apparatus 100, for example, according to an operation of an operator. For example, the hospital information system is a system for promoting efficient medical treatment and accounting work of a hospital in which the ultrasonic diagnostic apparatus 100 is installed and store subject information. A medical questionnaire collects subject information in order to stores information related to an examination when a subject receives a medical checkup. The medical questionnaire may be written on paper or stored in an electronic medium. The input interface 30 may be an optical character recognition (OCR) system in consideration of a case in which the medical questionnaire is on paper.

Subject information may be acquired from an ordering system, a radiology information system (RIS), an electronic medical record system, and the like having examination information which are equivalent to the hospital information system instead of the hospital information system. Subject information is used to obtain an operation candidate for the ultrasonic probe 10, for example. When the subject information is information based on a hospital system, the subject information may include information such as an examination purpose, an examination region, and an executed protocol provided by the hospital system.

Subject information stored in the hospital system includes, for example, items indicating subject characteristics such as “examination purpose,” “examination region,” and “executed protocol.” Subject information written on a medical questionnaire includes, for example, items such as “height,” “weight,” “BMI,” “blood pressure,” “body fat,” “sex,” “age,” “medical history,” “ethnic group (race),” “occupation,” “dietary life,” “drinking,” “smoking history,” “exercise habits,” “family medical history,” and in the case of a female subject, “birth history,” “first menstruation age,” “menopause age,” “menstruation state,” and “lactation.”

The input interface 30 in the present disclosure is not limited to physical operating parts such as a mouse and a keyboard. For example, electrical signal processing circuitry that receives an electrical signal corresponding to an input operation from an external input device provided separately from the apparatus and outputs this electrical signal to a control circuit is included in examples of the input interface 30. The output interface 40 may be provided in the ultrasonic diagnostic apparatus 100 or provided separately from the ultrasonic diagnostic apparatus 100.

The output interface 40 includes, for example, the display device 42, a speaker 44, a vibrator 46, and the like. The display device 42 is disposed at a position at which the display device 42 displays an image that can be visibly recognized by the operator, for example. The display device 42 displays an image based on information output from the ultrasonic diagnostic apparatus 100. The display device 42 presents an operation candidate for the ultrasonic probe 10 through the vision of the operator or the like. For example, the display device 42 may be a display or a projector that projects images.

The speaker 44 is disposed at a position at which the operator can hear sound, for example. The speaker 44 outputs sound based on information output from the ultrasonic diagnostic apparatus 100. The speaker 44 presents an operation candidate for the ultrasonic probe 10 through the sense of hearing of the operator or the like. The speaker 44 presents the operation candidate for the ultrasonic probe 10, for example, through strength of sound, a length of an interval, a tone level, and the like. For example, the speaker 44 may be provided in a headphone or an earphone worn by the operator. The vibrator 46 is provided, for example, at a position at which the operator can sense vibration. For example, the vibrator 46 is used by being attached to the body of the operator or being put in the clothes of the operator. The vibrator 46 vibrates in response to information output from the ultrasonic diagnostic apparatus 100. The vibrator 46 presents an operation candidate for the ultrasonic probe 10 through the sense of touch of the operator or the like. When the operation candidate for the ultrasonic probe 10 is presented through the sense of touch, for example, a method of adjusting a press resistance difference from a subject through the ultrasonic probe 10, or the like may be used.

The ultrasonic diagnostic apparatus 100 includes, for example, a communication interface 110, processing circuitry 120, and a memory 130. The processing circuitry 120 includes, for example, an image processing function 121, a first acquisition function 122, a second acquisition function 123, a generation function 124, and a presentation function 125. The processing circuitry 120 realizes these functions, for example, by a hardware processor executing programs stored in the memory 130.

The hardware processor refers to, for example, a circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). Programs may be directly incorporated in the circuit of the hardware processor instead of being stored in the memory 130. In this case, the hardware processor realizes functions by reading and executing the programs incorporated in the circuit thereof. The hardware processor is not limited to a configuration of a single circuit, and a plurality of independent circuits may be combined as a single hardware processor to realize each function. In addition, a plurality of components may be integrated into a single hardware to realize each function. The memory 130 may be a non-transitory storage medium (of hardware). The memory 130 stores apparatus information representing apparatus characteristics of the host apparatus, such as the type, model number, specifications, installation year, month and date, and production year, month and date of the ultrasonic diagnostic apparatus 100, as part of existing data.

The communication interface 110 includes, for example, a communication interface such as a network interface card (NIC). The communication interface performs communication of information with the ultrasonic probe 10, the state sensor 20, the input interface 30, and the output interface 40 in a wired manner or through a network. The communication interface 110 outputs received information to the processing circuitry 120. In addition, the communication interface 110 may transmit information to other devices connected in a wired manner or through the network under the control of the processing circuitry 120.

The communication interface 110 receives reflected wave information transmitted from the ultrasonic probe 10. The communication interface 110 receives probe state information output from the state sensor 20. The communication interface 110 transmits guide information generated by the processing circuitry 120 to the output interface 40.

The image processing function 121 in the processing circuitry 120 converts reflected wave information output from the ultrasonic probe 10 into image information to generate an ultrasonic image that is an image of the inside of a subject. The image processing function 121 stores information on the generated ultrasonic image in the memory 130. The image processing function 121 outputs the information on the generated ultrasonic image to the output interface 40. The display device 42 of the output interface 40 displays the ultrasonic image, for example. The ultrasonic image is used for the operator to diagnose a health condition of the subject or search for a lesion. The ultrasonic image may be a single image or a moving image having a plurality of continuously switching images.

The first acquisition function 122 acquires probe state information output from the state sensor 20, for example. When detection information that is a 3-axis acceleration and a 3-axis angular speed of the ultrasonic probe 10 detected by the 6-axis sensor 22 and a current value measured by the pressure sensor 24 is output from the state sensor 20, for example, the first acquisition function 122 calculates and acquires probe state information on the basis of the output detection information.

The probe state information acquired by the first acquisition function 122 is used to obtain an operation candidate for the ultrasonic probe 10. Further, the first acquisition function 122 stores the acquired probe state information in the memory 130 as part of the existing data. The memory 130 stores all probe state information stored in the past as part of the existing data. The ultrasonic diagnostic apparatus 100 accumulates the probe state information in the memory 130 as the existing data.

When the first acquisition function 122 stores the probe state information in the memory 130, the first acquisition function 122 associates the probe state information with the ultrasonic image stored by the image processing function 121 in the memory 130. For example, the probe state information may be associated with the ultrasonic image using a tag embedded in the ultrasonic image or stored in the memory 130 through a separate file associated with the ultrasonic image. When the ultrasonic image is a moving image, for example, the first acquisition function 122 may store probe state information at a timing when reflected wave information is received in the memory 130.

The second acquisition function 123 acquires subject information output from the input interface 30. When the operator performs diagnosis on the subject, the second acquisition function 123 acquires the subject information and acquires apparatus information stored in the memory 130. The second acquisition function 123 stores the acquired subject information in the memory 130 as part of the existing data. The memory 130 stores all subject information stored in the past as part of the existing data. The ultrasonic diagnostic apparatus 100 accumulates the subject information in the memory 130 as the existing data.

The generation function 124 generates an operation candidate for the ultrasonic probe 10 on the basis of the probe state information acquired by the first acquisition function 122 and the subject information and the apparatus information acquired by the second acquisition function 123. An operation candidate for the ultrasonic probe 10 is a candidate for an operation of changing the position or the orientation of the ultrasonic probe 10. Criteria for generating an operation candidate may be generated, for example, by a guideline creator or the like who operates the ultrasonic probe 10. The criteria for generating an operation candidate may be generated in the same manner as that for operation candidate data of a second embodiment which will be described later, for example.

The generation function 124 reads the existing data stored in the memory 130 at the time of generating an operation candidate for the ultrasonic probe 10. The generation function 124 generates the operation candidate for the ultrasonic probe 10 on the basis of the probe state information acquired by the first acquisition function 122, the subject information acquired by the second acquisition function 123, and the existing data read from the memory 130. The generation function 124 stores the generated operation candidate for the ultrasonic probe 10 in the memory 130 as part of the existing data. The memory 130 associates all operation candidates for the ultrasonic probe 10 stored in the past with probe state information and subject information and stores them as part of the existing data.

The presentation function 125 presents the operation candidate for the ultrasonic probe 10 generated by the generation function 124 to the operator. For example, the presentation function 125 outputs operation candidate information representing the operation candidate for the ultrasonic probe 10 to the output interface 40 at the time of presenting the operation candidate for the ultrasonic probe 10. The output interface 40 displays an image or outputs sound on the basis of the operation candidate information transmitted from the presentation function 125. When the output interface 40 includes the display device 42, for example, the display device 42 displays an operation candidate image in accordance with the operation candidate information output from the presentation function 125.

The subject information stored by the second acquisition function 123 and the existing data are accumulated in the memory 130. The existing data accumulated in the memory 130 is data according to probe state information and subject information collected by the ultrasonic diagnostic apparatus 100 at the time of past diagnoses. The existing data may include external data collected and accumulated by other ultrasonic diagnostic apparatuses 100 and other apparatuses. The external data may be data provided to the ultrasonic diagnostic apparatus 100 by external apparatuses other than the ultrasonic diagnostic apparatus 100. The ultrasonic diagnostic apparatus 100 may provide data collected thereby to an external apparatus or the like as external data. When the existing data is external data, the existing data includes apparatus information and the apparatus information is also accumulated.

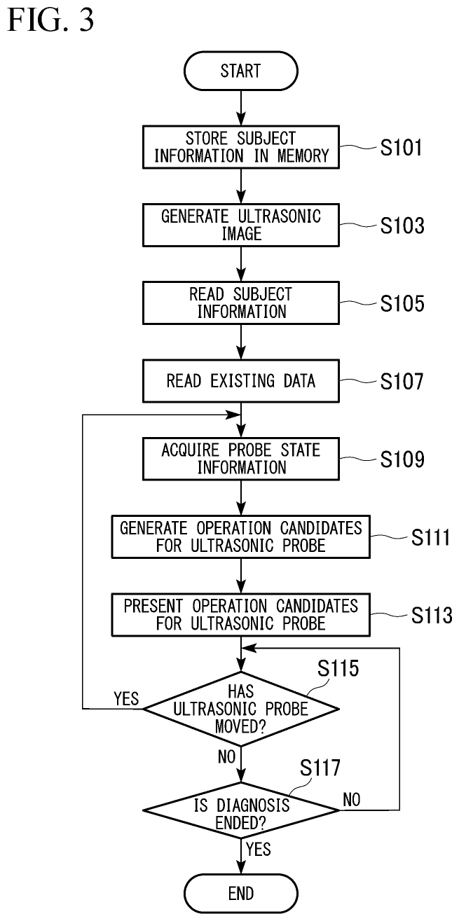

Next, processing of the ultrasonic diagnostic apparatus 100 of the first embodiment when diagnosis is performed on a subject will be described. FIG. 3 is a flowchart showing an example of processing of the ultrasonic diagnostic apparatus 100 of the first embodiment. At the time of performing diagnosis on a subject, for example, the operator inputs subject information of the subject through the input interface 30 as preprocessing of a diagnosis. The input interface 30 outputs the input subject information to the ultrasonic diagnostic apparatus 100. The ultrasonic diagnostic apparatus 100 acquires the subject information output from the input interface 30 through the second acquisition function 123 and stores the subject information in the memory 130 (step S101).

Subsequently, the ultrasonic diagnostic apparatus 100 generates an ultrasonic image on the basis of reflected wave information output from the ultrasonic probe 10 through the image processing function 121 (step S103). For example, the image processing function 121 marks an arbitrary position in the ultrasonic image generated by the image processing function 121 and sets the position as a specific position.

Subsequently, the second acquisition function 123 reads the subject information stored in the memory 130 (step S105). There are cases in which preprocessing of the diagnosis is not performed in step S101 and the subject information is not stored in the memory 130. In this case, the operator inputs the subject information through the input interface 30 and outputs the subject information to the ultrasonic diagnostic apparatus 100. The second acquisition function 123 acquires the subject information output from the input interface 30 instead of reading the subject information stored in the memory 130. Subsequently, the generation function 124 reads existing data accumulated in the memory 130 (step S107).

Subsequently, the first acquisition function 122 acquires probe state information, for example, a relative position, a scanning direction, a rotational direction, an inclination, and a pressing pressure of the ultrasonic probe 10 with respect to the subject on the basis of detection information output from the state sensor 20 (step S109). The first acquisition function 122 acquires the probe state information, for example, using the specific position specified by the image processing function 121 as a reference position. The reference position may be a position other than the specific position. For example, the reference position may be a position of an organ that is a diagnosis target or, if a position of a lesion is identified, the position of the lesion may be used as the reference position.

Subsequently, the generation function 124 integrates the probe state information acquired by the first acquisition function 122 and the subject information acquired by the second acquisition function 123 as acquired data and compares the acquired data with the existing data read from the memory 130. The generation function 124 generates an operation candidate for the ultrasonic probe 10 on the basis of a result of comparison between the acquired data and the existing data (step S111).

For example, the generation function 124 sets a target position, a target speed, and a target pressure with respect a state of the ultrasonic probe 10, such as a position, a speed, and a pressing pressure, on the basis of the existing data. For example, the generation function 124 compares the generated target values with the acquired data and generates an operation of the ultrasonic probe 10 by which a state of the ultrasonic probe 10 when the ultrasonic probe 10 is operated reaches the target values as an operation candidate for the ultrasonic probe 10. For example, the generation function 124 generates an operation candidate for the ultrasonic probe 10 for each of items such as a movement direction, a moving speed, a rotational direction, a rotating speed, and a pressing pressure of the ultrasonic probe 10. The generation function 124 generates an operation candidate for the ultrasonic probe 10 using a target pressure when the ultrasonic probe is pressed to the subject.

The generation function 124 may set target values of a state of the ultrasonic probe 10 through any method. For example, when there is variation in each item of probe state information in a plurality of pieces of existing data, the generation function 124 may set target values using an arithmetic operation result such as an average of each item or set the target values using existing data classified with reference to each item of the subject information. The generation function 124 may perform an arithmetic operation with respect to each item of the probe state information for each piece of existing data classified with reference to each item of the subject information and set target values of a state of the ultrasonic probe 10 using a result of the arithmetic operation.

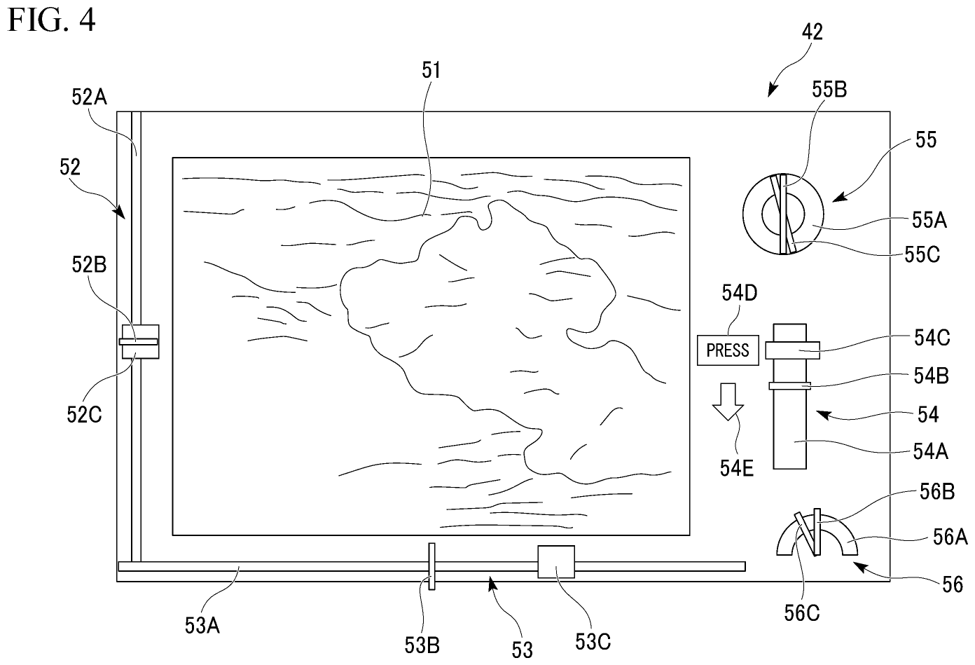

Subsequently, the presentation function 125 presents the operation candidate for the ultrasonic probe 10 generated by the generation function (step S113). The presentation function 125 outputs operation candidate information to the output interface 40 and causes the display device 42 of the output interface 40 to display an operation candidate image corresponding to the operation candidate information. As an image displayed on the display device 42, an image including the operation candidate image will be described. FIG. 4 is a diagram showing an example of a page displayed on the display device 42. In FIG. 4, it is assumed that the right direction of the page of the display device 42 is +X direction, the left direction is −X direction, the upward direction is +Y direction, and the downward direction is −Y direction.

For example, an ultrasonic image 51 generated by the image processing function 121 is displayed in the center area of the page of the display device 42. The ultrasonic image 51 is, for example, an image assumed to be visually recognized when a specific position is viewed at the position of the ultrasonic probe 10. As the ultrasonic image 51, an image viewed when the position of the head of the ultrasonic probe 10 is set to a viewpoint is displayed. Accordingly, the vertical direction and the horizontal direction in the ultrasonic image 51, for example, changes according to the orientation of the ultrasonic probe 10. The ultrasonic image may display a difference from an ultrasonic image generated in the past such as a previous ultrasonic image.

A height position indicator 52 is displayed on the −Y side of the ultrasonic image 51 and a horizontal position indicator 53 is displayed on the −X side of the ultrasonic image 51. A pressure indicator 54 is displayed at an approximately center position in the Y direction on the +X side of the ultrasonic image 51. A rotational position indicator 55 is displayed on the +Y side of the pressure indicator 54 and a speed indicator 56 is displayed on the −Y side of the pressure indicator 54.

The height position indicator 52 includes a display area image 52A, a target position image 52B, and a current position image 52C. Likewise, the horizontal position indicator 53 includes a display area image 53A, a target position image 53B, and a current position image 53C, and the pressure indicator 54 includes a display area image 54A, a target pressure image 54B, and a current pressure image 54C. The rotational position indicator 55 includes a display area image 55A, a target position image 55B, and a current position image 55C, and the speed indicator 56 includes a display area image 56A, a target speed image 56B, and a current speed image 56C.

The display area image 52A of the height position indicator 52 is displayed in a long area extending in the Y direction. The target position image 52B is displayed at an approximately center position of the display area image 52A in the Y direction. The current position image 52C can be displayed at any position on the display area image 52A. In the example shown in FIG. 4, the current position image 52C is superposed and displayed on the target position image 52B in the height position indicator 52. The height position indicator 52 indicates that a position in the height direction viewed from the ultrasonic probe 10 is consistent with a target position to the operator.

The display area image 53A of the horizontal position indicator 53 is displayed in a long area extending in the X direction. The target position image 53B is displayed at an approximately center position of the display area image 53A in the X direction. The current position image 53C can be displayed at any position on the display area image 53A. In the example shown in FIG. 4, the current position image 53C is displayed on the +X side further than the target position image 52B in the horizontal position indicator 53. The horizontal position indicator 53 indicates that a position in the horizontal direction viewed from the ultrasonic probe 10 is on the right side of a target position to the operator. Accordingly, the ultrasonic diagnostic apparatus 100 presents an operation of moving the ultrasonic probe 10 to the left to the operator.

The display area image 54A of the pressure indicator 54 is displayed in a long area extending in the Y direction. The display area image 54A of the pressure indicator 54 is shorter than the display area image 52A of the height position indicator 52. The target pressure image 54B is displayed at an approximately center position of the display area image 54A in the Y direction. The current pressure image 54C can be displayed at any position on the display area image 54A. In the example shown in FIG. 4, the current pressure image 54C is displayed on the +Y side further than the target pressure image 54B in the pressure indicator 54. The pressure indicator 54 indicates that a pressing pressure is less than a target pressure. Accordingly, the ultrasonic diagnostic apparatus 100 presents an operation of increasing (pressing) the pressing pressure to the operator.

The pressure indicator 54 further includes a pressing direction image 54D and a pressing instruction image 54E. The pressing direction image 54D indicates a direction in which a person performing diagnosis operates the ultrasonic probe 10. In the example shown in FIG. 4, the pressing direction image 54D indicates a direction in which the ultrasonic probe 10 is pressed to a subject side. The pressing instruction image 54E is information representing a state in which the ultrasonic probe 10 is operated. In the example shown in FIG. 4, the pressing direction image MD is represented as characters of “PRESS.” In this case, the pressure indicator presents an instruction for pressing the ultrasonic probe 10 to the person performing the diagnosis. When the pressing pressure is less than the target pressure, the pressing direction image 54D is represented as characters of “LIFT,” for example. In this case, the pressure indicator 54 presents an instruction for bringing back the ultrasonic probe 10 to the person performing the diagnosis. The presentation function 125 displays the target pressure image 54B as a pressure applied to the subject by the ultrasonic probe 10. The presentation function 125 displays the pressing direction image 54D as an orientation in which the ultrasonic probe 10 is operated. Information on the pressure applied to the subjected by the ultrasonic probe 10 and the orientation in which the ultrasonic probe 10 is operated may be generated by the presentation function or the generation function 124, for example. The pressing direction image 54D and the pressing instruction image 54E may be displayed in a superposed manner in the ultrasonic image 51. As the pressing direction image 54D, characters such as “MORE PRESSURE” and “APPLY MORE PRESSURE” instead of “PRESS!” may be displayed. Further, characters such as “LESS PRESSURE” and “APPLY LESS PRESSURE” instead of “LIFT!” may be displayed.

The display area image 55A of the rotational position indicator 55 is displayed in a circular area. The target position image 55B and the current position image 55C are displayed as any segments corresponding to a diameter connecting two points on the circumferential circle of the display area image 55A. In the example shown in FIG. 4, the target position image 55B is displayed as a segment in the Y-direction which corresponds to the diameter of the circular shape indicating the display area image 55A. The current position image 55C is displayed as a segment that corresponds to the diameter of the circular shape indicating the display area image 55A and rotates about 30 degrees from the target position image 55B counter clockwise. The rotational position indicator 55 indicates a counter clockwise deviation of about 30 degrees from a target rotation angle of the ultrasonic probe 10 to the operator. Accordingly, the ultrasonic diagnostic apparatus 100 presents an operation of rotating the ultrasonic probe 10 by about 30 degrees clockwise to the operator.

The display area image 56A of the speed indicator 56 is displayed in a semicircular area. The target speed image 56B is displayed at the center of the display area image 56A. The current speed image 56C can be displayed at any position on the display area image 56A. The speed indicator 56 indicates a higher speed when the positions at which the target speed image 56B and the current speed image 56C are displayed closer to the +X side. In the example shown in FIG. 4, the current speed image 56C is displayed on the −X side of the display area image 56A in the speed indicator 56. The speed indicator 56 indicates that the current speed is lower than a target speed of the ultrasonic probe 10 to the operator. Accordingly, the ultrasonic diagnostic apparatus 100 presents an operation of increasing the movement speed of the ultrasonic probe 10 to the operator. In the rotational position indicator 55 and the speed indicator 56, a direction image and an instruction image like the pressing direction image 54D and the pressing instruction image 54E in the pressure indicator 54 may also be displayed.

Referring back to the flowchart shown in FIG. 3, the ultrasonic diagnostic apparatus 100 determines whether the ultrasonic probe 10 has moved through the image processing function 121 (step S115). When the image processing function 121 determines that the ultrasonic probe 10 has moved, the ultrasonic diagnostic apparatus 100 returns to step S109 in which the first acquisition function 122 acquires probe state information. When the image processing function 121 determines that the ultrasonic probe 10 has not moved, the ultrasonic diagnostic apparatus 100 determines whether to end the diagnosis (step S117).

When it is determined that the diagnosis is not ended, the ultrasonic diagnostic apparatus 100 returns to step S115 and determines whether the ultrasonic probe 10 has moved through the image processing function 121. When it is determined that the diagnosis is ended, the ultrasonic diagnostic apparatus 100 ends processing of the flowchart shown in FIG. 3.

The above-described ultrasonic diagnostic apparatus 100 of the first embodiment generates an operation candidate for the ultrasonic probe on the basis of probe state information representing a relative relationship between the ultrasonic probe 10 and a subject and subject information representing subject characteristics of the subject and presents the operation candidate to the operator. Accordingly, the ultrasonic diagnostic apparatus 100 can present an appropriate operation of the ultrasonic probe 10 during a diagnosis to the operator. Therefore, the operator can perform appropriate diagnosis on the subject.

In addition, in a diagnosis using the ultrasonic probe 10 of the first embodiment, an appropriate pressing pressure is an important factor. However, the pressing pressure is adjusted mostly according to experience or the like of the operator, and thus it is difficult for the operator to press the ultrasonic probe 10 to a subject with an appropriate pressing pressure to perform diagnosis on the subject. In this respect, the ultrasonic diagnostic apparatus 100 of the first embodiment generates a target pressure with respect to a pressing pressure as well as a position and a speed of the ultrasonic probe 10 with respect to the operation of the ultrasonic probe 10. An operation candidate for the ultrasonic probe 10 is generated using this target pressure and a pressing pressure is presented to the operator. Accordingly, the operator can press the ultrasonic probe 10 to a subject with an appropriate pressing pressure.

Furthermore, at the time of presenting a target pressure, for example, it is conceived that a person performing a diagnosis cannot decide what kind of operation of the ultrasonic probe 10 is desirable even when the current pressure and the target pressure are simply displayed. In this respect, the ultrasonic probe 10 of the first embodiment presents the target pressure image 54B as a pressure applied to a subject by the ultrasonic probe and presents the pressing direction image 54D as an orientation in which the ultrasonic probe 10 is operated. Accordingly, it is possible to clearly present an appropriate operation to the person performing a diagnosis.

In addition, the ultrasonic diagnostic apparatus 100 of the first embodiment generates an operation candidate for the ultrasonic probe 10 on the basis of probe state information and subject information accumulated as existing data. Accordingly, the operation candidate for the ultrasonic probe 10 can be generated in a relation between the state of the ultrasonic probe 10 and the subject, and thus an appropriate operation of the ultrasonic probe 10 can be presented to the operator.

Furthermore, the ultrasonic diagnostic apparatus 100 of the first embodiment updates and presents an operation candidate for the ultrasonic probe whenever the ultrasonic probe 10 moves with the progress of diagnosis. Accordingly, even when the progress of diagnosis has changed from a schedule, for example, an appropriate operation of the ultrasonic probe 10 can be presented to the operator.

Moreover, the ultrasonic diagnostic apparatus 100 of the first embodiment includes the display device 42, the speaker 44, and the vibrator 46 as the output interface 40 and presents an operation candidate for the ultrasonic probe 10 through the vision, the sense of hearing, or the sense of touch of the operator. Accordingly, it is possible to present the operation candidate for the ultrasonic probe 10 to the operator without depending on a diagnostic situation of the operator.

### Second Embodiment

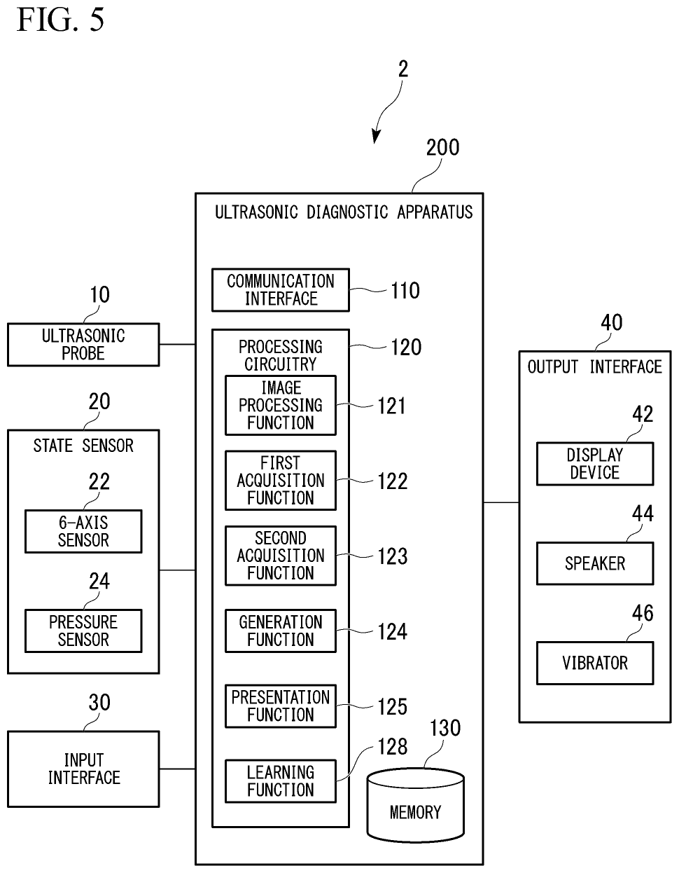

FIG. 5 is a block diagram of an ultrasonic diagnostic system 2 of the second embodiment. As shown in FIG. 5, in the ultrasonic diagnostic system 2 of the second embodiment, the processing circuitry 120 in an ultrasonic diagnostic apparatus 200 includes a learning function 128. The learning function 128 generates a trained model that outputs data (hereinafter referred to as “operation candidate data”) of an operation candidate for the ultrasonic probe 10 by receiving acquired data. In the ultrasonic diagnostic apparatus 200 of the second embodiment, the generation function 124 functions after the trained model is generated. The generation function 124 uses the trained model that outputs the operation candidate data by receiving acquired data. The generation function 124 generates the operation candidate data that is an operation candidate for the ultrasonic probe 10 by inputting acquired data acquired by the first acquisition function 122 and the second acquisition function 123 to the trained model. Other components are the same as those of the ultrasonic diagnostic system 1 of the first embodiment.

For example, the acquired data includes data of probe state information, subject information, and apparatus information acquired by the first acquisition function 122 and the second acquisition function 123. The acquired data and the operation candidate data are training data when a trained model is generated, the acquired data is input data, and the operation candidate data is output data. The training data may be collected and accumulated in the ultrasonic diagnostic apparatus 200 or collected and accumulated by an external apparatus or the like including a separately provided learning system and acquired by the ultrasonic diagnostic apparatus 200. The external apparatus may be installed in a facility in which the ultrasonic diagnostic apparatus 200 is installed or installed in another facility. When the external apparatus is installed in another facility, the ultrasonic diagnostic apparatus 200 may receive a trained model transmitted from the external apparatus in a wired or wireless manner.

The training data may not be data collected and accumulated through diagnoses performed by the operator. For example, the training data may be data generated using probe state information and an operation candidate for the ultrasonic probe 10 acquired by operating the ultrasonic probe 10 in a case other than a case where a reference operator for generating reference data performs diagnosis on a subject. In this case, subject information and apparatus information may be arbitrarily conceived information. For example, the subject information and the apparatus information may be information collected and accumulated as in the above-described first embodiment.

The learning function 128 generates a trained model, for example, when the generation function 124 has generated an operation candidate for the ultrasonic probe on the basis of acquired data. The trained model is stored in the memory 130, for example, and the learning function 128 reads the trained model stored in the memory 130 and updates the read trained model to generate a new trained model at the time of generating a trained model.

A trained model includes, for example, an input layer, a hidden layer, and an output layer. The learning function 128 reads a trained model stored in the memory 130 at the time of generating a trained model. For example, the learning function 128 receives acquired data acquired by the first acquisition function 122 and the second acquisition function 123 and outputs operation candidate data from the output layer through the hidden layer.

The hidden layer includes a multi-layered neural network that connects the input layer and the output layer. Parameters of the hidden layer is optimized, for example, by performing machine learning such as deep learning using the acquired data input to the input layer and the operation candidate data output from the output layer. The learning function 128 stores the generated trained model along with the training data in the memory 130.

Next, processing executed in the ultrasonic diagnostic apparatus 200 of the second embodiment will be described. In the second embodiment, phases in the ultrasonic diagnostic apparatus 200 are divided into two phases: a learning phase; and a normal diagnosis phase. The learning phase is executed prior to the normal diagnosis phase. The learning phase is a phase in which a trained model is generated. The normal diagnosis phase is a phase in which diagnosis is performed on a subject. For example, the learning phase may be the feedback of a phase executed simultaneously with the normal diagnosis phase executed in the past.

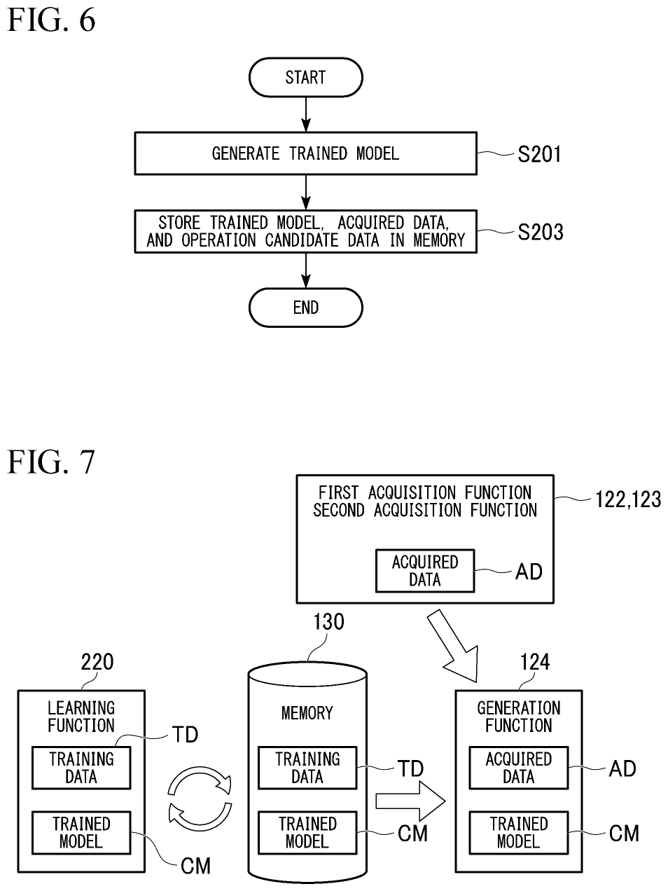

First, the learning phase will be described. FIG. 6 is a flowchart showing an example of processing of the ultrasonic diagnostic apparatus 200 of the second embodiment. In the ultrasonic diagnostic apparatus 200, the learning function 128 generates a trained model according to machine learning when the generation function 124 has generated operation candidate data, as shown in FIG. 6 (step S201). Subsequently, the learning function 128 stores the generated trained model along with training data in the memory 130 (step S203). Thereafter, the ultrasonic diagnostic apparatus 200 ends processing of the flowchart shown in FIG. 6.

Next, the normal diagnosis phase will be described. In the normal diagnosis phase, processing is executed through the same flow as processing executed in the ultrasonic diagnostic apparatus 100 of the first embodiment shown in FIG. 3. Differences from processing in the ultrasonic diagnostic apparatus 100 of the first embodiment are primarily described. In a process of generating an operation candidate for the ultrasonic probe 10 in step S113, the generation function 124 reads a trained model stored in the memory 130. The generation function 124 inputs acquired data to the input layer of the read trained model. The generation function 124 generates operation candidate data output from the output layer of the trained model as an operation candidate for the ultrasonic probe 10. Other processes are performed in the same manner as processes executed in the ultrasonic diagnostic apparatus 100 of the first embodiment.

FIG. 7 is a conceptual diagram showing a data flow through which the ultrasonic diagnostic apparatus 200 performs machine learning and executes a normal diagnosis. As shown in FIG. 7, in the learning phase, the learning function 128 reads training data TD and a trained model CM stored in the memory 130 when the generation function 124 has generated an operation candidate for the ultrasonic probe 10. Subsequently, the learning function 128 updates the trained model CM read from the memory 130 using acquired data obtained by the first acquisition function 122 and the second acquisition function 123, learning candidate data generated by the learning function 124, and the training data TD read from the memory 130 to generate a trained model CM. The learning function 128 stores the generated trained model CM in the memory 130 as a new trained model CM.

In the normal diagnosis phase, the generation function 124 reads the trained model CM stored in the memory 130 when the first acquisition function 122 and the second acquisition function 123 have obtained acquired data. Subsequently, the generation function 124 generates an operation candidate for the ultrasonic probe 10 using the acquired data AD obtained by the first acquisition function 122 and the second acquisition function 123 and the trained model CM read from the memory 130.

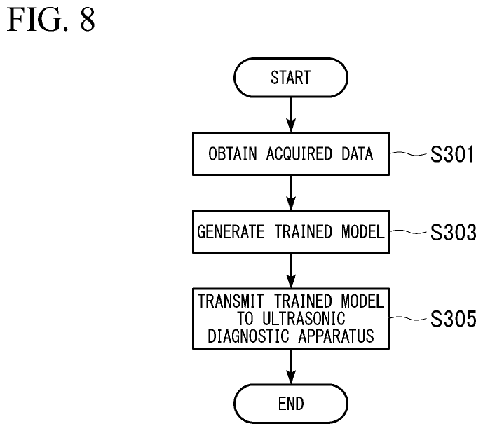

In the second embodiment, there are cases in which a trained model is generated in an external apparatus. Processing in the external apparatus when the trained model is generated in the external apparatus will be described with reference to FIG. 8. FIG. 8 is a flowchart showing an example of processing of the external apparatus. As shown in FIG. 8, the external apparatus obtains acquired data including probe state information, subject information, and apparatus information from a plurality of apparatuses including the ultrasonic diagnostic apparatus 200 which perform diagnoses using the ultrasonic probe (step S301). Subsequently, the external apparatus generates a trained model according to machine learning using training data including the obtained acquired data (step S303). Subsequently, the external apparatus transmits the generated trained model to the ultrasonic diagnostic apparatus 100 (step S305). In this manner, the external apparatus ends processing of the flowchart shown in FIG. 8.

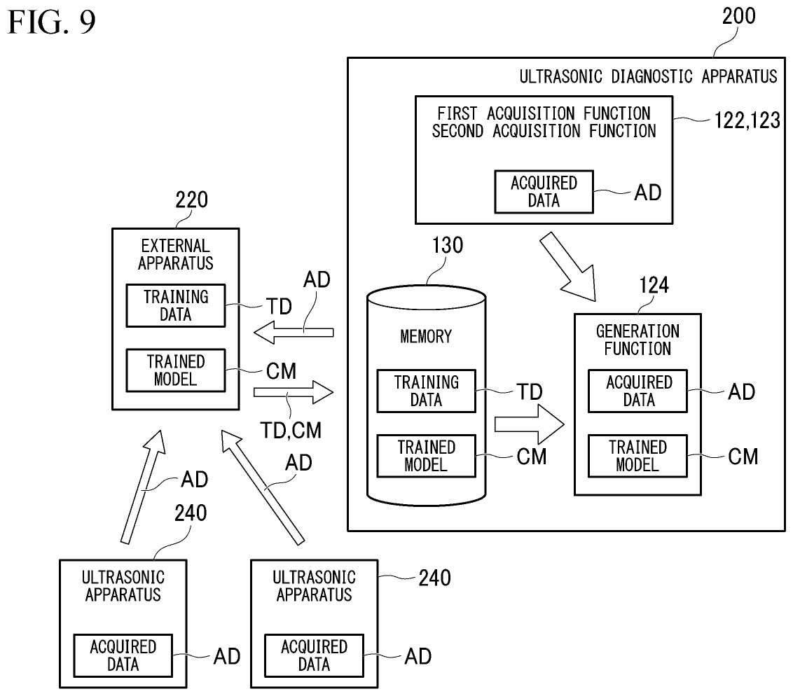

FIG. 9 is a conceptual diagram showing a data flow through which an external apparatus 220 performs machine learning and the ultrasonic diagnostic apparatus 200 executes a normal diagnosis. In this example, the external apparatus 220 is a learning system which stores training data and a trained model and updates the training data and the trained model to generate a trained model. Acquired data AD is transmitted to the external apparatus 220 from the ultrasonic diagnostic apparatus 200 and a plurality of ultrasonic apparatuses 240 other than the ultrasonic diagnostic apparatus 200. The ultrasonic apparatuses 240 are apparatuses that obtain the acquired data AD and may be an ultrasonic diagnostic apparatus or an apparatus other than the ultrasonic diagnostic apparatus.

As shown in FIG. 9, the external apparatus 220 receives the acquired data AD transmitted from the ultrasonic diagnostic apparatus 200 and the ultrasonic apparatuses 240. The external apparatus 220 updates a trained model CM using the received acquired data AD and training data TD stored therein to generate a trained model. The external apparatus 220 transmits the generated trained model CM to the ultrasonic diagnostic apparatus 200. The ultrasonic diagnostic apparatus 200 stores the received trained model CM in the memory 130. The trained model CM is updated and generated at any time in the external apparatus 220 by repeating this procedure. The generation function 124 in the ultrasonic diagnostic apparatus 200 reads the trained model CM from the memory 130 when an ultrasonic diagnosis is performed on a subject. The generation function 124 generates operation candidate data using the read trained model CM.

The above-described ultrasonic diagnostic apparatus 200 of the second embodiment generates an operation candidate for the ultrasonic probe 10 using a trained model generated by performing machine learning using accumulated acquired data. Accordingly, it is possible to generate an operation close to a proper operation as an operation candidate for the ultrasonic probe 10 with high accuracy.

### Third Embodiment

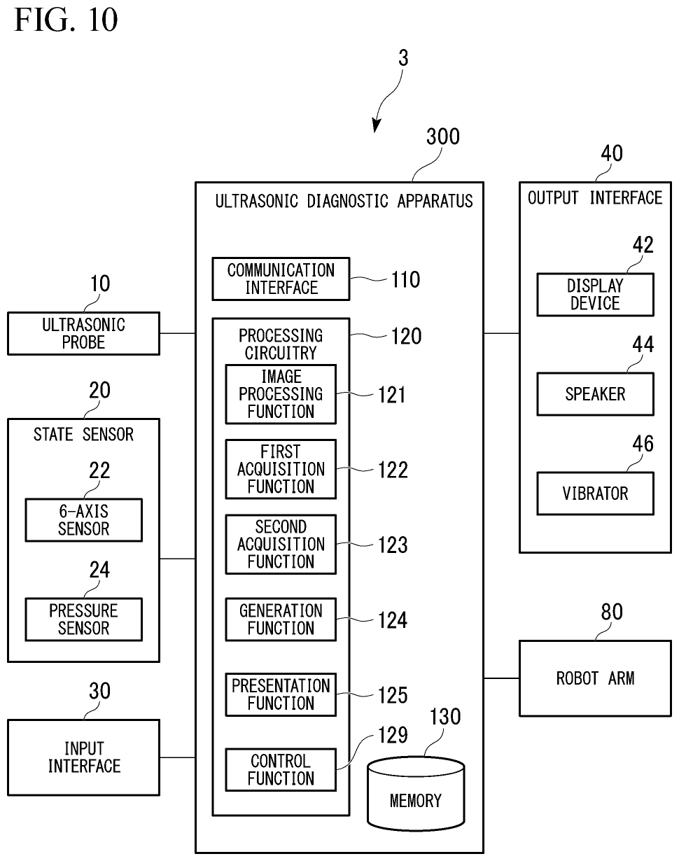

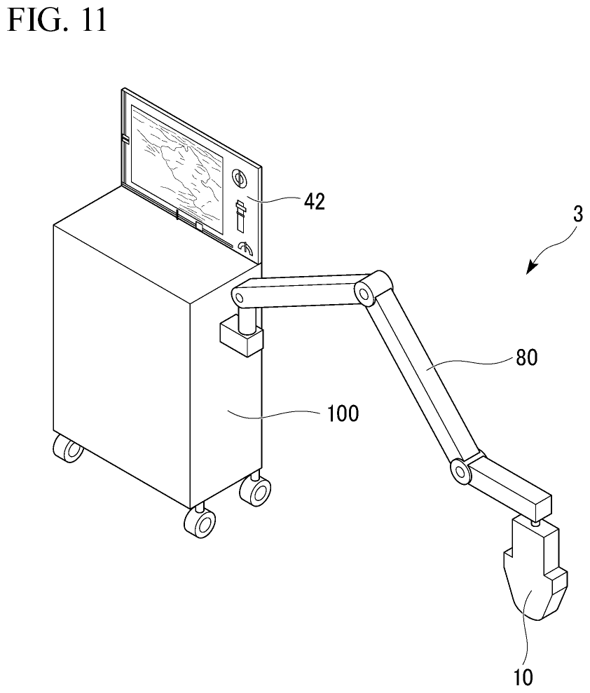

FIG. 10 is a block diagram of an ultrasonic diagnostic system 3 of the third embodiment and FIG. 11 is a diagram showing the appearance of the ultrasonic diagnostic system 3. As shown in FIG. 10, in the ultrasonic diagnostic system 3 of the third embodiment, the processing circuitry 120 in an ultrasonic diagnostic apparatus 300 includes a control function 129. The ultrasonic diagnostic apparatus 300 includes a robot arm 80. Other components are the same as those of the ultrasonic diagnostic system 1 of the first embodiment.

As shown in FIG. 11, the robot arm 80 is attached to the housing of the ultrasonic diagnostic apparatus 300. The robot arm 80 is, for example, so-called a 6-axis robot and can move in 3-axis directions and rotate on 3 axes. The ultrasonic probe 10 is attached to the tip of the robot arm 80, for example. The robot arm 80 includes a control mechanism for operating the ultrasonic probe 10. The 6-axis sensor 22 and the pressure sensor 24 are provided in the robot arm 80, for example.

The control function 129 controls, for example, the operation of the robot arm 80. The presentation function 125 determines a candidate state of the ultrasonic probe 10 for a subject on the basis of an operation candidate for the ultrasonic probe generated by the generation function 124. The control function 129 acquires a current probe state of the ultrasonic probe 10 on the basis of probe state information and subject information detected by the 6-axis sensor 22 and the pressure sensor 24. The control function 129 calculates a difference between the candidate state of the ultrasonic probe 10 and the current probe state of the ultrasonic probe 10. The control function 129 operates the robot arm 80 such that the calculated difference is eliminated and the state of the ultrasonic probe 10 becomes the candidate state. The ultrasonic diagnostic apparatus 300 presents an operation candidate for the ultrasonic probe 10 according to operation of the robot arm 80.

The ultrasonic diagnostic apparatus 300 performs diagnosis on a subject by operating the ultrasonic probe 10 through the robot arm 80. Accordingly, diagnosis is performed on the subject without an operator holding and operating the ultrasonic probe 10. For example, the operator performs input processing on the input interface 30 in the ultrasonic diagnostic apparatus 300. Alternatively, the ultrasonic diagnostic apparatus 300 performs diagnosis on the subject thereby without depending on the operation of the operator.

The above-described ultrasonic diagnostic apparatus 300 of the third embodiment performs diagnosis on a subject by operating the ultrasonic probe 10 using the robot arm 80 and thus can perform diagnosis on the subject even when an operator is inexperienced in diagnosis or an operator is absent. In the ultrasonic diagnostic apparatus 300 of the third embodiment, the control function 129 controls the robot arm 80 that is a control mechanism for operating the ultrasonic probe 10 and adjusts the state of the ultrasonic probe 10 on the basis of a candidate state of the ultrasonic probe 10 determined by the presentation function 125 to perform diagnosis on the subject. Accordingly, the ultrasonic diagnostic apparatus 300 can perform appropriate diagnosis on the subject.

Although the output interface 40 is provided in the third embodiment, the output interface 40 may not be provided. In this case, the presentation function 125 may determine a candidate state of the robot arm 80 and may not perform generation and output of operation candidate information for the output interface 40.

### Other Examples

For example, a computer aided diagnosis (CAD) function may be provided in the above-described ultrasonic diagnostic apparatuses 100, 200, and 300. In the CAD function, for example, the image processing function 121 extracts feature quantities from a generated ultrasonic image and performs image analysis. The image processing function 121 may cause the display device 42 to display a result of image analysis instead of the ultrasonic image, for example. In the image analysis, for example, the feature quantities of the generated ultrasonic image are compared with known feature quantities to calculate difference degrees therebetween. Subsequently, the calculated difference degrees are classified on the basis of a predetermined threshold value. The known feature quantities include, for example, feature quantities selected through a learning process of machine learning with respect to feature quantities of collected ultrasonic images.

In the CAD function, image analysis is performed to determine the presence or absence of a lesion for ultrasonography performed in a state in which the ultrasonic probe 10 is pressed to a subject with a specific pressing pressure. The specific pressing pressure applied to the subject by the ultrasonic probe 10 is determined, for example, by a reference operator who generates training data using machine learning of the CAD function. This specific pressing pressure is stored in the memory 130 along with the training data used for machine learning of the CAD function.

In the ultrasonic diagnostic apparatus 100 including the CAD function, when it is determined whether a subject has a lesion, a target pressure may be generated and an operation candidate for the ultrasonic probe 10 in response to the target pressure may be presented as described in each of the above-described embodiments at the time of pressing the ultrasonic probe 10 to the subject. When the ultrasonic diagnostic apparatus 100 includes the control function 129 and the robot arm 80 as in the third embodiment, the control function 129 may control the robot arm 80 in response to an operation candidate for the ultrasonic probe 10.

Although one target value of an operation of the ultrasonic probe 10 is set and presented with respect to each item and an operation candidate for the ultrasonic probe 10 is presented in each of the above-described embodiments, a plurality of target values may be set and presented according to predetermined conditions. For example, in the ultrasonic diagnostic apparatus including the aforementioned CAD function, a target pressure (recommended pressure) that becomes a target (recommended) may be displayed according to the type of the CAD function.

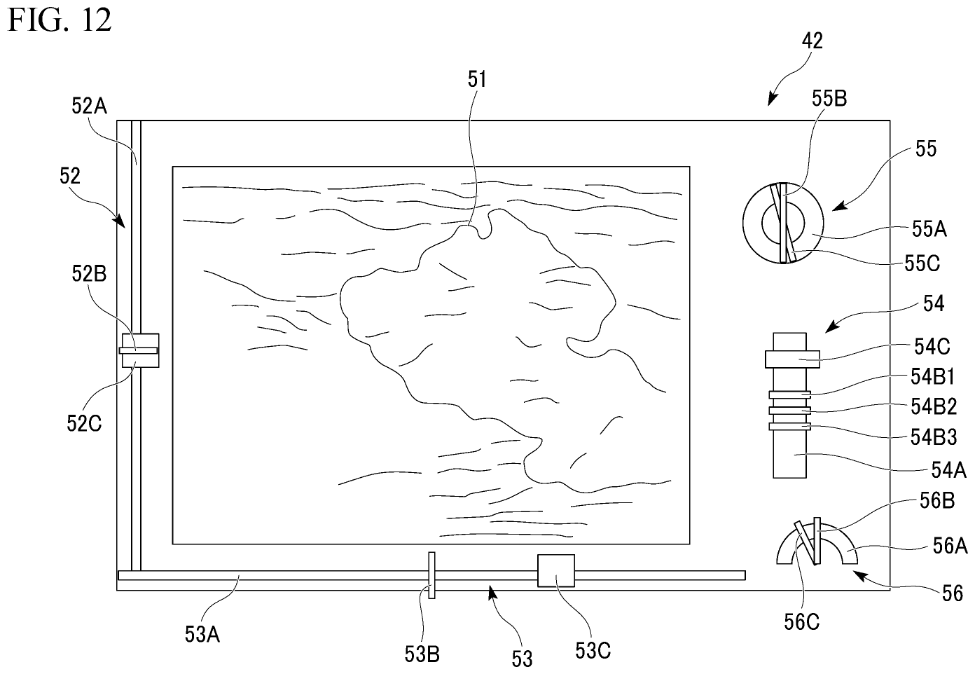

FIG. 12 is a diagram showing an example of a page displayed on the display device 42. FIG. 12 shows an example of a plurality of target pressures in the ultrasonic diagnostic apparatus including the aforementioned CAD function. As shown in FIG. 12, a first target pressure 54B1, a second target pressure 54B2, and a third target pressure 54B3 are displayed on the display device 42 as target pressures.

The first target pressure 54B1 is a target pressure recommended by a first CAD function. The second target pressure 54B2 is a target pressure recommended by a second CAD function. The third target pressure 54B3 is a target pressure recommended by a third CAD function. The first to third CAD functions may be functions included in the same or same type of ultrasonic diagnostic apparatuses or functions included in different types of ultrasonic diagnostic apparatuses. The first target pressure 54B1, the second target pressure 54B2, and the third target pressure 54B3 may be displayed for each CAD function that has collected training data. In this case, only a target pressure according to a CAD function that has collected training data may be displayed.

In this manner, recommended pressures (target pressures) according to a plurality of CAD functions may be simultaneously displayed in the ultrasonic diagnostic apparatus. The ultrasonic diagnostic apparatus may display a plurality of target values with respect to other items, for example, a relative position, scanning direction, a rotational direction, and an inclination of the ultrasonic probe 10 with respect to a subject as target values of a state of the ultrasonic probe 10.

According to at least one of the above-described embodiments, it is possible to perform appropriate diagnosis on a subject by including processing circuitry configured to convert a signal into image information, the signal being generated by an ultrasonic probe receiving reflected waves of ultrasonic waves which have been transmitted from the ultrasonic probe and reflected from a subject, acquire information representing a relative relationship of the ultrasonic probe with respect to the subject, acquire at least one of information representing subject characteristics of the subject and information representing apparatus characteristics of the ultrasonic diagnostic apparatus, and generate an operation candidate for the ultrasonic probe on the basis of the acquired information representing the relative relationship of the ultrasonic probe with respect to the subject and the acquired at least one of the information representing the subject characteristics and the information representing the apparatus characteristics.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processing circuitry configured to:
convert a signal into image information, the signal being generated by an ultrasonic probe receiving reflected waves of ultrasonic waves which have been transmitted from the ultrasonic probe and reflected from a subject;
acquire information representing a relative relationship of the ultrasonic probe with respect to the subject;
acquire at least one of information representing subject characteristics of the subject and information representing apparatus characteristics of the ultrasonic diagnostic apparatus; and
generate an operation candidate for the ultrasonic probe on the basis of the acquired information representing the relative relationship of the ultrasonic probe with respect to the subject and the acquired at least one of the information representing the subject characteristics and the information representing the apparatus characteristics.

2. The ultrasonic diagnostic apparatus according to claim 1, wherein the processing circuitry is configured to:
generate the operation candidate for the ultrasonic probe using a target pressure when the ultrasonic probe is pressed to the subject; and
generate information presenting a pressure to be applied to the subject by the ultrasonic probe and an orientation in which the ultrasonic probe is to be operated.

3. The ultrasonic diagnostic apparatus according to claim 1, wherein the processing circuitry is configured to:
generate the operation candidate for the ultrasonic probe on the basis of accumulated relative relationships of the ultrasonic probe with respect to the subject.

4. The ultrasonic diagnostic apparatus according to claim 1, wherein the processing circuitry is configured to:
generate the operation candidate for the ultrasonic probe on the basis of at least one of accumulated subject characteristics and apparatus characteristics.

5. The ultrasonic diagnostic apparatus according to of claim 1, wherein the processing circuitry is configured to:
update the operation candidate for the ultrasonic probe with the progress of a diagnosis.

6. The ultrasonic diagnostic apparatus according to claim 1, wherein the processing circuitry is configured to:
present the operation candidate for the ultrasonic probe.

7. The ultrasonic diagnostic apparatus according to claim 6, wherein the processing circuitry is configured to:
present the operation candidate for the ultrasonic probe through at least one of vision, a sense of hearing, and a sense of touch of an operator.

8. The ultrasonic diagnostic apparatus according to claim 1, wherein the processing circuitry is configured to:
control a control mechanism for operating the ultrasonic probe on the basis of the operation candidate for the ultrasonic probe.

9. The ultrasonic diagnostic apparatus according to claim 1, wherein the operation candidate for the ultrasonic probe is information generated using a model generated according to machine learning using the acquired information representing the relative relationship of the ultrasonic probe with respect to the subject, the acquired at least one of the information representing the subject characteristics and the information representing the apparatus characteristics, and information on the operation candidate for the ultrasonic probe as training data.

10. An ultrasonic diagnostic system comprising:
an ultrasonic probe which transmits ultrasonic waves and receives reflected waves of the transmitted ultrasonic waves; and
the ultrasonic diagnostic apparatus according to claim 1.
